(19) United States
(12) Patent Application Publication
(10) Pub. No.: US 2017/0258409 A1
(43) Pub. Date: Sep. 14, 2017

(54) SYNCHRONIZED MULTI-MODULE PULSED ARTERIAL SPIN LABELED MAGNETIC RESONANCE IMAGING

(71) Applicant: UNIVERSITY OF SOUTHERN CALIFORNIA, Los Angeles, CA (US)

(72) Inventors: Hung Phi Do, Los Angeles, CA (US); Terrence Jao, Los Angeles, CA (US); Krishna S. Nayak, Los Angeles, CA (US)

(21) Appl. No.: 15/529,054

(22) PCT Filed: Dec. 1, 2015
(86) PCT No.: PCT/US15/63226

§ 371 Date: May 23, 2017

Related U.S. Application Data
(60) Provisional application No. 62/085,912, filed on Dec. 1, 2014.

(51) Int. Cl. A61B 5/00; G01R 33/567; G01R 33/563; A61B 5/026; A61B 5/055

(52) U.S. Cl. CPC A61B 5/7285; A61B 5/0263; A61B 5/055; G01R 33/563; G01R 33/5673

(57) ABSTRACT

A magnetic resonance imaging system may include a magnet, gradient coils, an RF pulse transmitter, an RF receiver that receives MR signals from tissue that has been exposed to RF pulses, gradient fields, and a magnetic field, and a computer that includes a processor. The computer may have a configuration that: causes the RF pulse transmitter and gradient coils to emit multiple labeling pulses at predetermined labeling times directed to blood in a subject; causes the RF pulse transmitter, gradient coils, and magnet to generate MR signals directed to tissue at one or more spatial locations within the subject that receives the blood; causes the RF receiver to receive MR signals emitted by the tissue at predetermined imaging times; generates an image of the tissue based on the received MR signals; repeats the foregoing four actions one or more times; and generates information indicative of perfusion within the tissue based on the generated images.

### CROSS-REFERENCE TO RELATED APPLICATION

This application is based upon and claims priority to U.S. provisional patent application 62/085,912, entitled “MULTI-PULSED ARTERIAL SPIN LABELED MAGNETIC RESONANCE IMAGING,” filed Dec. 1, 2014, attorney docket no. 094852-0046. The entire content of this application is incorporated herein by reference.

### BACKGROUND

### Technical Field

This disclosure relates to Arterial spin labeling (ASL), which may use a magnetic resonance imaging (MRI) apparatus to generate images of perfusion (tissue blood flow) non-invasively, without administering contrast agents to the subject.

### Description of Related Art

MRI is a technique that may magnetically excite nuclear spins in a subject under a magnetic field with radio frequency (RF) pulses at the Larmor frequency using an RF transmitter. Images may be obtained from excitation induced free-induction decay (FID) signals using an RF receiver.

The ASL technique may be used to apply an RF pulse by which the magnetization spin state of blood is labeled, and when the bolus of labeled blood reaches the imaging slice is subjected to image acquisition. Two images may be generated using the ASL technique: one labeled image and one control image. The control image may be taken under the same conditions as the labeled image without labeling. The two images may be subjected to a pixel difference operation to obtain an ASL image that is indicative of perfusion, which is inflowing blood in the microcirculation.

In the ASL technique, perfused blood may only contribute 1-4% of the measured MR signals. That may make ASL an extremely low signal-to-noise ratio (SNR) technique. ASL may be subjected to a pixel difference operation between a labeled and a control image. That may make ASL sensitive to subject movement and physiological motions that may occur during or between imaging acquisitions of labeled and control images. In the ASL technique, the time it takes labeled blood to reach tissue that receives blood may be called arterial transit time. The received MR signals from the ASL technique may also exponentially decay over time at a time constant called a longitudinal relaxation time or T₁ (that may be 1-2 seconds at 3 Tesla). That may reduce the amplitude of the MR signals of the ASL technique if arterial transit times are long (>2 sec). Arterial transit times may be long under conditions of collateral blood flow, disease states that reduce blood flow velocity, and large spatial separation between a labeling location of blood and a imaging location of tissues, which may occur when imaging locations are sequential multiple slices, simultaneous multiple slices, or a 3D volume.

### SUMMARY

A magnetic resonance imaging system may include a magnet, gradient coils, an RF pulse transmitter, an RF receiver that receives MR signals from tissue that has been exposed to RF pulses, gradient fields, and a magnetic field, and a computer that includes a processor. The computer may have a configuration that: causes the RF pulse transmitter and gradient coils to emit multiple labeling pulses at predetermined labeling times directed to blood in a subject; causes the RF pulse transmitter, gradient coils, and magnet to generate MR signals directed to tissue at one or more spatial locations within the subject that receives the blood; causes the RF receiver to receive MR signals emitted by the tissue at predetermined imaging times; generates an image of the tissue based on the received MR signals; repeats the foregoing four actions one or more times; and generates information indicative of perfusion within the tissue based on the generated images.

The magnetic imaging system may include a sensor that monitors a physiological activity of the subject. The predetermined labeling times and predetermined imaging times may be set by the computer based on the monitored physiological activity of the subject.

The physiological activity may be heartbeats and the predetermined labeling times may be set by the computer to occur during a user-specified cardiac phase within each heartbeat.

The predetermined labeling times and the predetermined imagine times may be set by computer to occur every heartbeat.

The predetermined labeling times may be set by computer to occur when the RF receiver receives MR signals emitted by the tissue.

The labeling pulses and imaging may continuously occur.

The acquired MR signals may be retrospectively processed based on recorded physiological activity.

The physiological activity may be respiration. The predetermined labeling and imaging times may be set by the computer based on a rate of the monitored respiration of the subject.

The physiological activity may be blood flow. The predetermined labeling times may be set by the computer to occur when the monitored blood flow has a rate above a threshold.

The sensor may provide an ECG, PG, acoustic, optical, electromagnetic, real-time MR, CT, PET, SPECT, or ultrasound navigator signal in response to the monitored physiological activity. The computer may set the labeling and imaging times in real time based on the signal provided by the sensor.

The predetermined labeling and imaging times may be specified by a human operator.

The predetermined imaging times may be set by the computer to occur immediately following completion of the multiple labeling pulses.

The multiple labeling pulses may be of different user-selectable types.

The different user-selectable types of pulses may include spatially selective, velocity selective, and acceleration selective pulses.

The computer may cause the RF pulse transmitter, gradient coils, and magnet to generate MR signals directed to the tissue at multiple spatial locations simultaneously.

The computer may cause the RF pulse transmitter, gradient coils, and magnet to generate MR signals directed to the tissue at multiple spatial locations sequentially.

The computer may cause the RF pulse transmitter, gradient coils, and magnet to generate MR signals directed to the tissue at multiple spatial locations in different organs.

The predetermined labeling times may be both before and between the predetermined imaging times.

The computer may generate information indicative of arterial transit time from the location of the blood to which the labeling pulses are directed to the spatial locations of tissue that receives the blood based on the images.

These, as well as other components, steps, features, objects, benefits, and advantages, will now become clear from a review of the following detailed description of illustrative embodiments, the accompanying drawings, and the claims.

### BRIEF DESCRIPTION OF DRAWINGS

The drawings are of illustrative embodiments. They do not illustrate all embodiments. Other embodiments may be used in addition or instead. Details that may be apparent or unnecessary may be omitted to save space or for more effective illustration. Some embodiments may be practiced with additional components or steps and/or without all of the components or steps that are illustrated. When the same numeral appears in different drawings, it refers to the same or like components or steps.

FIG. 1 illustrates an example of a magnetic resonance imaging (MRI) system.

FIG. 2 is a flow chart that shows an example of an MRI process.

FIG. 3 is a schematic diagram of one possible setting of SYMPASL.

FIGS. 4A, 4B, 4C, 4D, and 4E each show sequence timings that may be used in an MRI process. The process in FIG. 4B may be used to acquire sequential multiple slices; the process in FIG. 4C may be used to acquire 3D volumetric coverage; the process in FIG. 4D may be used to simultaneously acquire multiple slices (SMS); and the process in FIG. 4E may be used to acquire the same imaging section multiple times.

FIG. 5 illustrates a labeling and imaging plan that may be used in an MRI process.

FIG. 6 illustrates a simplified sequence timing that may be used for quantification of perfusion.

FIG. 7 shows a saturation efficiency map from a representative subject.

FIGS. 8A, 8B, and 8C show SYMPASL results on a kidney from a representative subject in comparison with simulation and with FAIR.

FIG. 9 shows SNR efficiency measured from SYMPASL and FAIR in comparison with simulation data.

### DETAILED DESCRIPTION OF ILLUSTRATIVE EMBODIMENTS

Illustrative embodiments are now described. Other embodiments may be used in addition or instead. Details that may be apparent or unnecessary may be omitted to save space or for a more effective presentation. Some embodiments may be practiced with additional components or steps and/or without all of the components or steps that are described.

MRI systems will now be described that may improve SNR and SNR efficiency and reduce sensitivity to arterial transit time and physiological motions. The MRI systems may enable volumetric perfusion imaging in the heart and other organs.

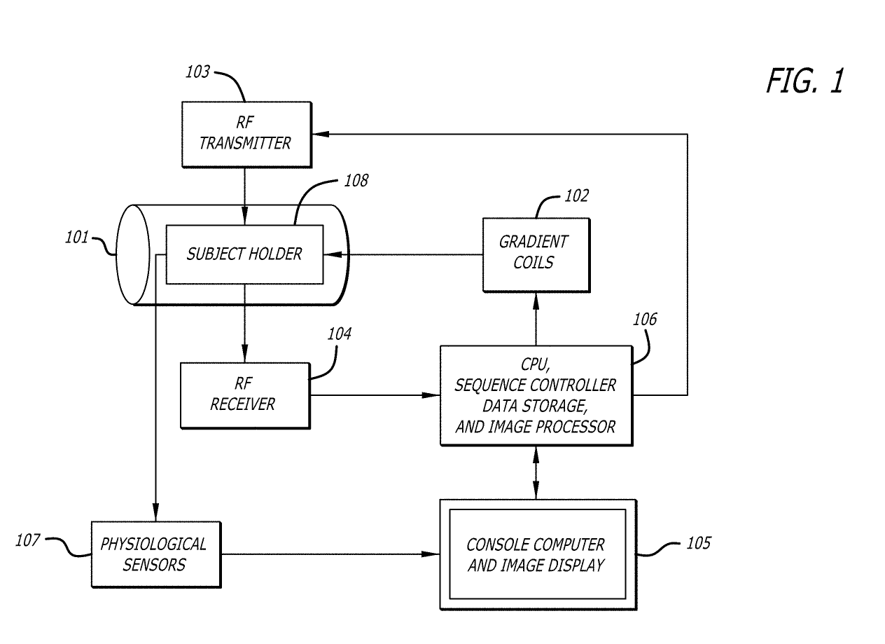

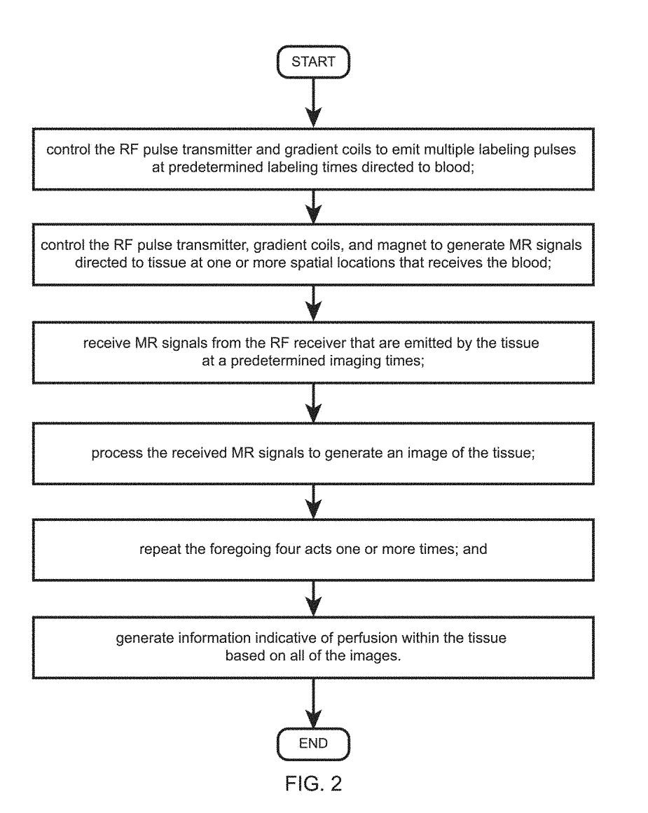

FIG. 1 illustrates an example of a magnetic resonance imaging (MRI) system. FIG. 2 is a flow chart that shows an example of an MRI process.

The magnetic resonance imaging (MRI) system in FIG. 1 may perform the techniques disclosed in this specification, including the process disclosed in FIG. 2.

A magnet 101 may polarize a subject contained within a subject holder 108. Three orthogonal gradient coils 102 may localize labeling and imaging. An RF transmitter 103 may label blood and may excite the target subject for imaging. An RF receiver 104 may receive MR signal that are emitted by the target tissue. A computer 105 may include a processor 106, may control the MR pulse sequence, and may process MR signals to generate information indicative of perfusion within tissue of interest. One or more physiological sensors 107 may monitor physiological activity of the subject to control the timing of labeling and imaging. The sensors may include ECG, PG, acoustic detector, optical sensor, electromagnetic sensor, motion sensor, real-time MR, CT, PET, SPECT, or ultrasound navigator and may, respectively, monitor the heart rate, blood flow waveform, tissue motion, and respiratory motion of the subject. The computer 105 may be configured to cause the various components of the MRI system to automatically perform the functions described herein and to itself perform the image generation steps described herein after the MRI system is activated.

Arterial spin labeling (ASL) is a contrast-free magnetic resonance based imaging technique that may be capable of measuring tissue perfusion. The ASL technique may include two steps: labeling followed by imaging. Described herein is a new and efficient labeling and imaging scheme for ASL called physiologically Synchronized Multi-module Pulsed Arterial Spin Labeled (SYMPASL) MRI.

A labeling method called physiologically Synchronized Multi-module Pulsed Arterial Spin Labeled (SYMPASL) may involve applying several spatially selective RF pulses proximal to the target tissue e.g. imaging slab. Repeating the labeling pulse for multiple times may ensure that there will be some ASL signal even in cases where the arterial transit time is large (>2 s). Furthermore, using several labeling pulses may provide a higher ASL signal than when using a single labeling pulse because a larger amount of blood may be labeled.

The timing of the labeling and imaging may be determined based on the pulsatile flow pattern of feeding arteries and cardiac motion, if needed. The proposed labeling scheme may possess several advantages in comparison to an ideal labeling scheme. These may include one or more of the following: (a) compatibility with volumetric imaging including 3D imaging, simultaneous multi-slice imaging, and sequential multi-slice imaging; (b) compatibility with real time cardiac triggering making it less sensitive to cardiac motion (which may be one of dominant sources of physiological noise); and (c) lower sensitivity to arterial transit time which may be problematic, especially in the patient population.

In general, SYMPASL can be compatible with volumetric perfusion imaging of almost any organs, including the brain, heart, kidneys, and lungs. Beside blood flow measurement, SYMPASL may also be able to measure arterial transit delay. This may also be another indicator of physiopathology. Another potential application is to non-contrast angiography (i.e. vessel imaging).

SYMPASL may provide high labeling efficiency and may be compatible with volumetric imaging. Furthermore, SYMPASL may be compatible with pulsatile flow and real-time cardiac gating. These features of SYMPASL may allow it to achieve myocardial perfusion imaging ASL with whole-heart coverage. In other organs where flow nature of feeding artery may be pulsatile, SYMPASL may provide labeling efficiency comparable to state-of-the-art labeling schemes which may be pseudo continuous arterial spin labeling (PCASL).

SYMPASL may be particularly useful for the assessment of cardiac perfusion defects related to coronary artery disease. Coronary artery disease (CAD) may be the single leading cause of death in the US that may be responsible for approximately 500,000 deaths per year, and decreased quality of life for nearly 16 million Americans. Myocardial perfusion imaging (MPI) may play an important role in accurately diagnosing of CAD.

Current technologies may have one or more of several issues, including invasive, using radiation, using exogenous contrast agent, low sensitivity, and not repeatable. SYMPASL may become a diagnostic and screening tool of CAD, because this MRI-based technique may be safe, repeatable, and may not require the use of an exogenous contrast agent. This may have an immediate impact in 615,000 Americans with end-stage renal disease (ESRD) who need frequent screening of CAD every 12 months.

SYMPASL may utilize multiple labeling pulses that may be timed based on pulsatile flow pattern of the feeding artery. It may improve labeling efficiency and sensitivity compared to current ASL implementations. It may also be compatible with whole-heart coverage myocardial perfusion imaging.

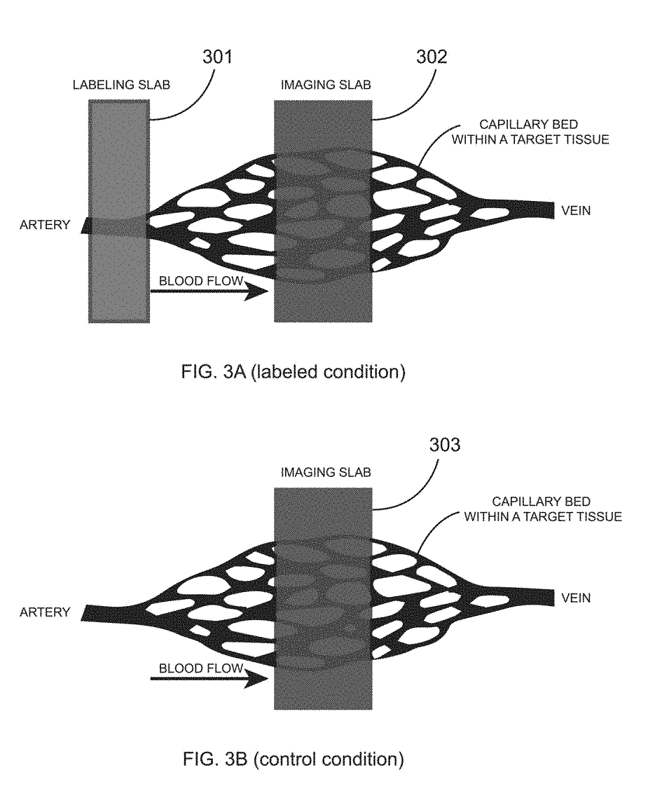

FIG. 3 is a schematic diagram of one possible setting of SYMPASL. Multiple RF pulses 301 may be applied to label upstream blood. A “labeled” image 302 may be acquired either immediately after the last labeling pulse or at any predetermined delay time. The labeled image may reflect signal from labeled blood delivered to target tissue. A second (“control”) image 303 may also be acquired in the absence of the labeling pulses. The difference image may be directly proportional to blood flow to the target tissue. A combination of labeling and imaging with appropriate timing can measure tissue perfusion of target tissue and may have units of ml-blood per g-tissue per min (ml/g/min). The time it takes the labeled blood to reach the target tissue may be called arterial transit time.

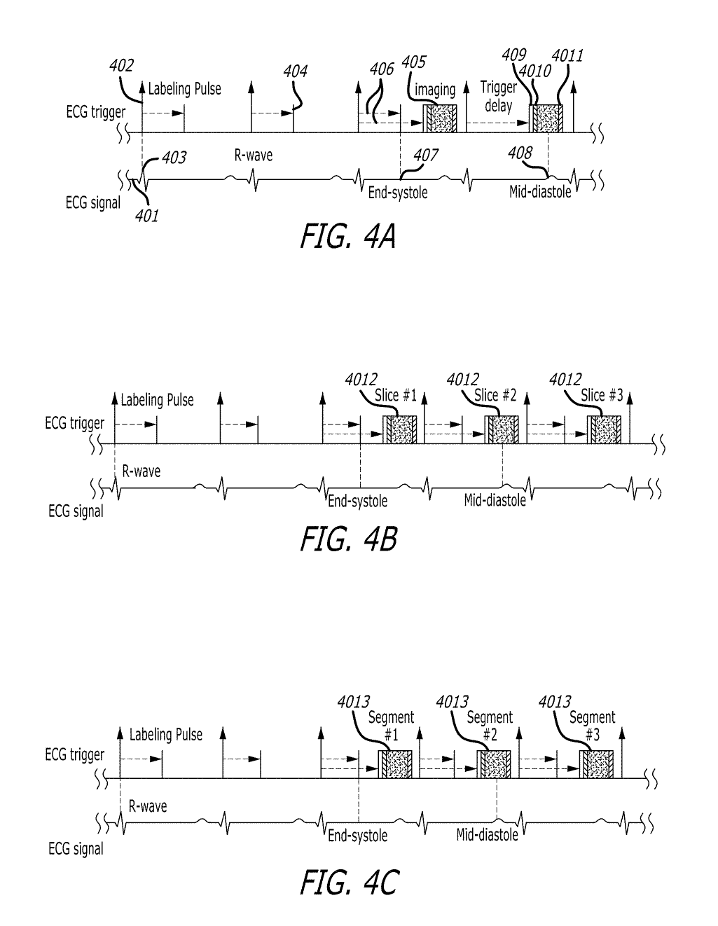

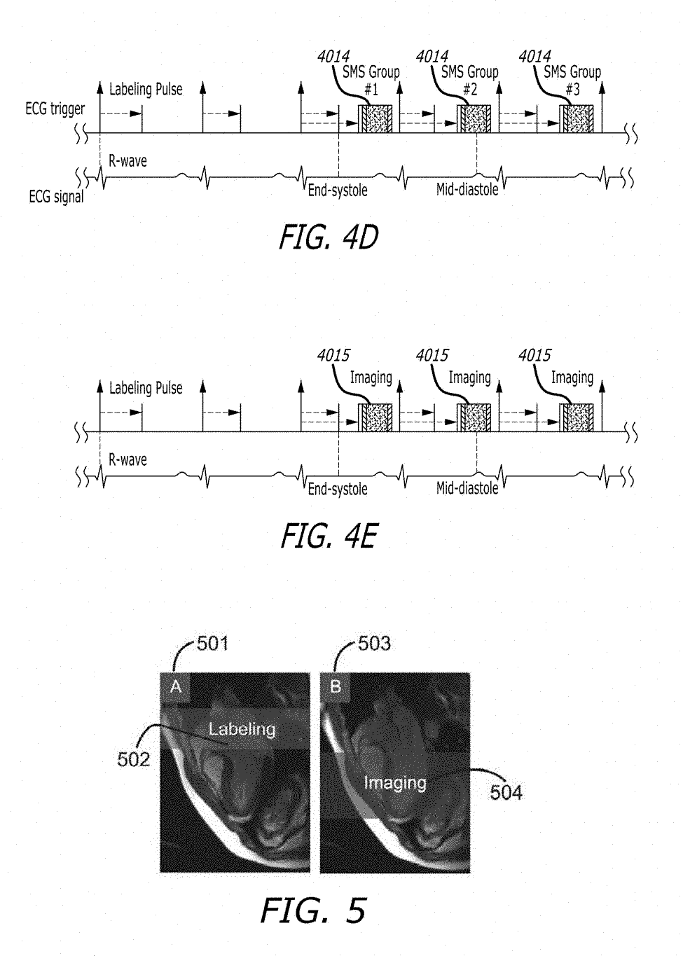

FIGS. 4A, 4B, 4C, 4D, and 4E each show sequence timings that may be used in an MRI process. The process in FIG. 4B may be used to acquire sequential multiple slices; the process in FIG. 4C may be used to acquire 3D volumetric coverage; the process in FIG. 4D may be used to simultaneously acquire multiple slices (SMS); and the process in FIG. 4E may be used to acquire the same imaging section multiple times.

In one application, SYMPASL may be used in the heart. SYMPASL may involve applying several spatially selective RF pulses to label blood in the proximal aorta at end systole for multiple consecutive heartbeats, as shown in FIG. 4A. Repeating the labeling pulse for multiple heartbeats may ensure that there will be some ASL signal, even in cases where the coronary transit time is larger than 1 heartbeat. In this example, the design may be based on the pulsatile nature of coronary flow. During systole, there may be minimal or no coronary blood flow due to ventricular contraction. Coronary flow may occur mainly during diastole when the coronaries are under low pressure. Placing the labeling pulse at the closure of the aortic valve may efficiently “label” all blood that is going to the coronary artery each cardiac cycle. In an additional example, the labeling scheme may possess several advantages as an ideal labeling scheme because it may be: (a) compatible with whole-heart imaging including 3D imaging, simultaneous multi-slice imaging, and sequential multi-slice imaging; (b) compatible with real time cardiac triggering as indicated by dash arrows in FIG. 4A, making it less sensitive to cardiac motion (which may be a dominant source of temporal variation in myocardial ASL); and (c) less sensitive to arterial transit delay.

FIG. 4A shows SYMPASL sequence timing for the heart. ECG signal 401 is measured from the subject. Solid arrows are ECG triggers 402 detected by the MRI scanner that corresponds to the R-wave 403 (highest peak of ECG signal) of the ECG signal. ECG triggers may be used to prospectively time the labeling pulses 404 and imaging 405, as indicated by dashed line arrows 406 (e.g. trigger delay). In this specific example, 10 ms RF labeling pulses 404 may be applied at end systole 407 to label blood at the proximal aorta and 150 ms imaging acquisitions 405 may be applied at mid-diastole 408, where the cardiac motion is the least. Fat saturation RF pulse 409 may be applied to reduce contribution of fat signal; ramp-up 4010 and ramp-down 4011 RF pulses may be applied before and after imaging acquisition to stabilize the acquired MR signal.

FIGS. 4B, 4C, 4D, and 4E show possible sequence timing for SYMPASL that may allow imaging of multiple slices 4012, a 3D volume 4013, groups of simultaneous multiple slices (SMS) 4014, and the same imaging section multiple times 4015, respectively.

In another example, SYMPASL may include labeling pulses. As such, 1D saturation or inversion of a thick slab covering the proximal aorta may be used with a geometry shown in FIG. 5. The precise timing, labeling, and imaging locations may be decided based on an ECG-gated thee-chamber CINE scout scan that may be used to set trigger delays and place the labeling and imaging volume. Pulsed labeling may occur at the time of aortic valve closure (i.e. at end-systole) (panel A), and imaging may occur at mid-diastole (panel B).

FIG. 5 illustrates an SYMPASL labeling and imaging plan. This planning may be based on a 3-chamber CINE image series that may be available in cardiac exams. Panel A shows a 3-chamber view image at end-systole 501 with a 1D labeling slab 502 placed at the proximal aorta. Panel B is the same view, but at mid-diastole 503, with an imaging region 504 that covers the entire left ventricular myocardium.

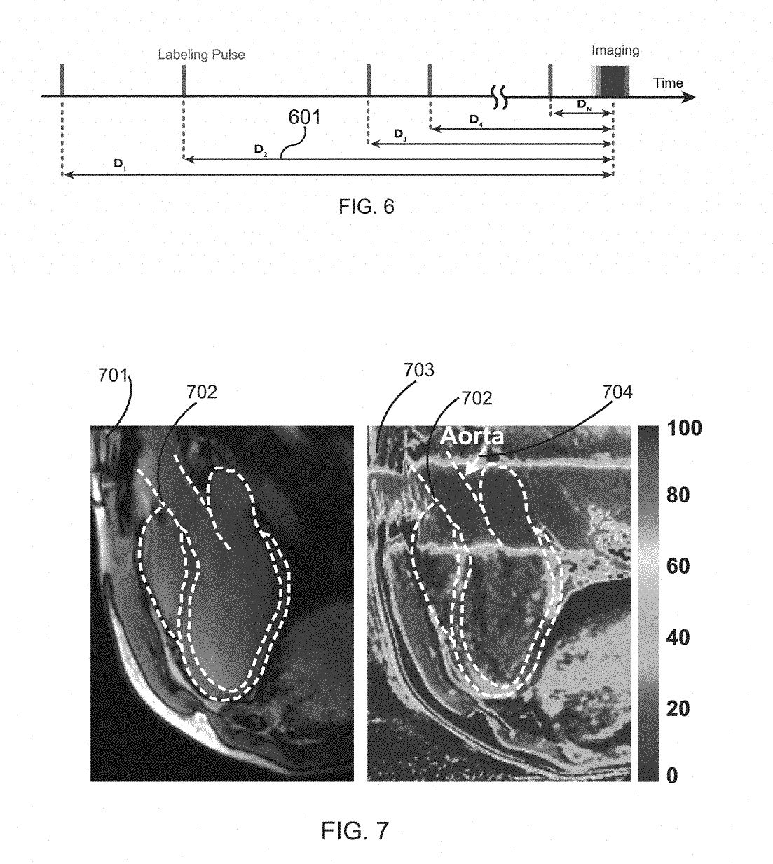

Blood Flow F in units of ml/g/min may be quantified using equations [1] and [2] where there is no overlap between labeled bolus and there are fractions of flow overlap fₖ between labeled boluses, respectively:

$$\begin{matrix}F = \frac{C - L}{α \cdot λ \cdot B \cdot {Σ}_{k = 1}^{N} ( {β}_{k} - δ ) \cdot exp ( - \frac{{D}_{k}}{{T}_{1}} )} , & [ 1 ] \\ F = \frac{C - L}{α \cdot λ \cdot B \cdot {Σ}_{k = 1}^{N} ( {β}_{k} - δ ) \cdot ( 1 + {f}_{k} ) \cdot exp ( - \frac{{D}_{k}}{{T}_{1}} )} , & [ 2 ]\end{matrix}$$

where C, L, and B refer to signal intensity in the control, labeled, and baseline images, respectively. α is labeling efficiency, which may be 1 and 2 for saturation and inversion pulse respectively. A is the blood-tissue-density. βₖ (with k=1,2,3 . . . N) is the bolus widths of the k^{th} labeling pulse. δ is the arterial transit time. Dₖ 601 is the delay time from the k^{th} labeling pulse to the center of the imaging window, as shown in FIG. 6, where N is the total number of labeling pulses. T₁ is longitudinal relaxation time of blood. fₖ is fraction of flow overlap between consecutive boluses.

In another example, SYMPASL may include sequence optimization. As such, the number of labeling pulses and the timing of imaging can be optimized for various desirable features such as ASL signal and ASL signal per unit time. The optimization may be specific for the target tissue because the flow pattern or feeding arteries may be different with different organs.

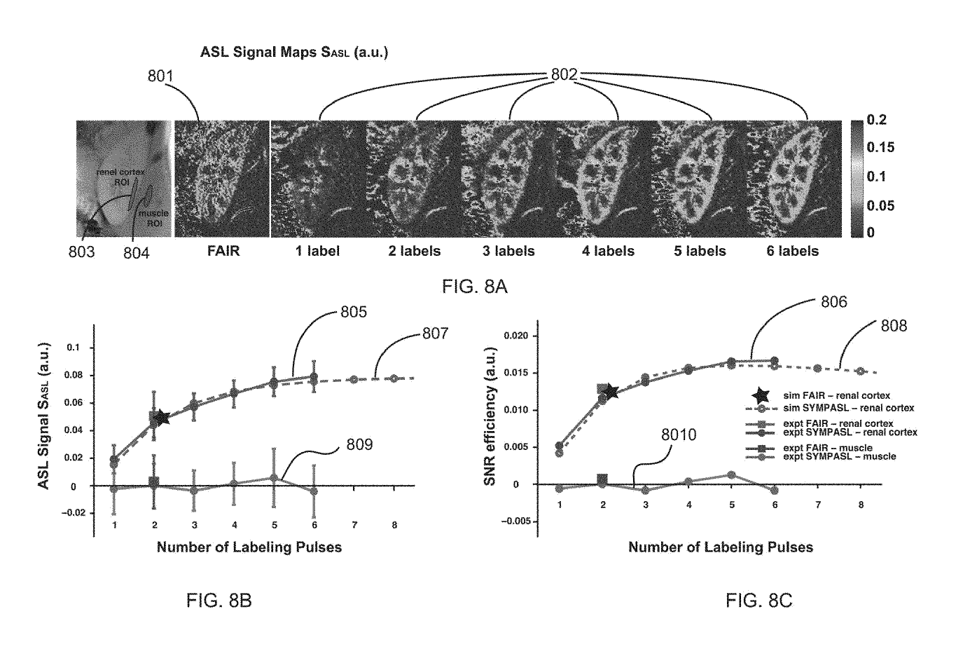

The use of saturation pulses for labeling may make the labeling independent of spin history and may eliminate any sensitivity to valvular heart disease (e.g. aortic regurgitation). FIGS. 8A-8C demonstrate a performance of the tailored saturation (TSAT) pulses. The tailored saturation pulses may be insensitive to B1 and B0 field inhomogeneity and may provide excellent performance in vivo. Results show consistently good saturation efficiency of >95%.

FIG. 7 shows saturation labeled efficiency measured in a representative subject. The left panel shows a 3-chamber view 701 with dashed white lines 702 to outline the heart. The right panel shows the corresponding labeling efficiency 703 measured using a tailored saturation pulse. The arrow 704 indicates the region of interest for labeling (e.g. the proximal aorta). Measured efficiency of >95% can be achieved in vivo.

In another example, inversion or saturation can be used as labeling pulses, depending on application and organs. The labeling pulse may be spatially selective and insensitive to off-resonance and B1+variation. Sung K et al., Measurement and characterization of RF nonuniformity over the heart at 3T using body coil transmission, J. Magn. Res. Imag. 2008; 27(3):643-648 suggested that the labeling pulses must tolerate up to ±120 Hz of off-resonance and up to 30% variations in B1+scale, at 3 Tesla. A number of possible labeling pulse designs can be used including Shinnar-Le Roux slab, optimal-control RF design. Adiabatic pulses or tailored saturation pulses can be incorporated for more robust performance to B0 and B1 inhomogeneity. The spatially selective RF pulses may be 1D, 2D, and 3D.

SYMPASL may also be compatible with the use of velocity selective (VS) labeling. Instead of spatial selection, VS labeling pulses may label blood based on its velocity. The VS labeling pulse may label (saturate) blood that is moving faster than a certain pre-determined velocity (V_{c}: cut-off velocity), while leaving slower moving blood untouched. Timing of the VS labeling pulse may be crucial to optimally labeled blood, which may be pulsatile. Similarly, SYMPASL may also be compatible with acceleration selective labeling in which blood is labeled based on its acceleration, rather than velocity or spatial location.

SYMPASL may be suitable for myocardial blood perfusion imaging to diagnose CAD. It may be a non-invasive, safe, and repeatable imaging technique for CAD patients, especially those with CKD and ESRD.

SYMPASL may have several advantages over current state-of-the-art myocardial ASL methods. ASL methods may be (a) compatible with volumetric imaging, (b) insensitive to arterial transit delay, (c) high ASL signal efficiency, (d) compatible with pulsatile flow which is ubiquitous in arteries, (e) insensitive to field inhomogeneity, (d) insensitive to physiological motion, and (e) independent of artery orientation. SYMPASL may be applied in most organs, including the brain, the kidneys, the lungs, and the heart. In cardiac imaging, the MRI imaging approaches that have been discussed may enable a study of the entire heart muscle.

Compatible with volumetric imaging (general property): Arterial transit time may be the main issue that prevents volumetric imaging. SYMPASL utilizes several labeling pulses at consecutive heartbeats. This labeling scheme may ensure that labeled blood will reach all parts of the target tissue, even areas with arterial transit time >1-2 s. Long arterial transit time may require a long delay time (typically delay time>=arterial transit time) to ensure labeled blood has reached the target tissue. This may lead to reduced ASL signal in PASL, where only a single labeling pulse may be used. As a result, SYMPASL may be compatible with volumetric imaging, including 3D imaging, simultaneous multi-slice imaging, and sequential multi-slice imaging.

Compatible with pulsatile flow (general property): Arterial blood flow may be pulsatile, meaning that the flow may mainly occur during a small fraction of the cardiac cycle. As a result of pulsatile flow in the feeding arteries, most of the blood delivery may also occur during a small fraction of the cardiac cycle. SYMPASL labeling pulses may be timed such that it labels blood just before the rise in flow. This labeling strategy may be efficient for pulsatile flow that is ubiquitous in feeding arteries of most organs, including the brain, the heart, the kidneys, and the lungs. SYMPASL may offer compatible labeling efficiency with the most efficient labeling method PCASL, which is flow dependent.

Insensitive to arterial transit delay (general property): SYMPASL may utilize several labeling pulses at consecutive heartbeats. It may ensure that labeled blood will reach the tissue of interest, even in areas with arterial transit delays that are >2 s (e.g. collateral flow and distal tissue from the feeding artery).

Improved SNR and SNR efficiency (general property): Compared to ASL methods with a single labeling pulse, SYMPASL may provide higher ASL signal due to accumulation of labeled blood from multiple pulses. ASL may be an inherently low SNR method because the perfused blood signal may be approximately 1-4% of tissue signal. ASL images may be acquired N times for averaging to compensate for low SNR. Compared to the FAIR implementation SYMPASL may have a N-fold increase in number of averages because any difference image between control and labeled images can be for averaging.

Insensitive to cardiac motion (general property): In the proposed pulse sequence, both labeling and imaging may be triggered to happen at a certain cardiac phase. Therefore, the proposed method may be relatively insensitive to cardiac motion. Unlike SYMPASL, the FAIR general kinetic model (FAIR-Buxton) may rely on the fact that both control and labeled images must have the same inversion time (TI) i.e. the delay time between labeling and imaging. This restriction may not allow acquisition window of the 2^{nd} image to be real-time cardiac triggered. This may make FAIR-Buxton sensitive to heart rate variation and cardiac motion. Another implementation of FAIR named FAIR-Apparent-T₁ may not require Tls of control and labeled images to be the same. Therefore, FAIR-Apparent-T₁ may also be insensitive to heart rate variation and cardiac motion. However, it may be less widely used compared to FAIR-Buxton, due to less efficiency.

Insensitive to the presence or absence of valvular disease (when saturation labels are used): In patients with valvular disease (e.g. aortic regurgitation), labeled blood at the proximal aorta may travel back to the left ventricular blood pool after labeling and may be pumped to the proximal aorta in the next heartbeat where it may be labeled by the consecutive labeling pulse. The second pulse may undo the effect of the first labeling pulse and may reduce labeling efficiency if inversion pulses are used. Saturation may be independent of spin history. Therefore, it can eliminate any sensitivity to valvular disease. Additionally, saturation can have flexible spacing in time as well as arbitrary bolus width regardless of flow pattern and velocity.

Low RF energy deposition (compared to PCASL): In PCASL, a train of RF pulses may be used as a labeling module. The RF pulse train may last 1-2 s, which may lead to high RF energy deposition into the patient (i.e. high specific absorption rate (SAR)). Unlike PCASL, SYMPASL may use 1-5 RF pulses of 10-20 ms in duration that each result in significantly lower SAR. SAR issues may be particularly of concern at high field imaging (>3T).

Insensitive to field inhomogeneity (compared to PCASL): PCASL labeling module may be sensitive to B₀ and B₁ magnetic field inhomogeneity. The SYMPASL labeling pulses may be either adiabatic or tailored, which may be insensitive to field inhomogeneity. The field inhomogeneity may be particularly problematic at high field imaging (>3T).

Insensitive to artery orientation (compared to PCASL): The labeling plane of PCASL may need to be prescribed such that it is perpendicular to the feeding arteries in order to achieve optimal performance. It may require an additional scout scan to facilitate localization. The labeling pulse in SYMPASL may be a thick slab that is independent of flow orientation and may therefore be insensitive to artery orientation.

In short, SYMPASL may have one or more of the following advantages over state-of-the-art myocardial ASL methods:

SYMPASL may be compatible with whole-heart imaging.

SYMPASL may be less sensitive to arterial transit delay.

SYMPASL may be insensitive to cardiac motions and heart rate variability.

SYMPASL with saturation labeling may be insensitive to the presence of valvular disease.

In pulsatile flow settings (in brain, kidney, and etc.), SYMPASL can provide comparable SNR to PCASL, which may be the highest efficiency labeling method to date.

SYMPASL may deposit less radiofrequency energy into the patient compared to PCASL. Furthermore, SYMPASL may be less sensitive to magnetic field inhomogeneity. These features may make it potentially superior to PCASL, especially at high field MRI where RF energy deposition and field inhomogeneity are issues.

In another example, SYMPASL may be applied to the kidney. FIG. 8 contains simulation and experimental data from one representative subject where FIG. 8A shows S_{ASL} maps from FAIR 801 and SYMPASL 802 with varying number of labeling pulses from 1 to 6. FIGS. 8B and 8C show S_{ASL} and SNR efficiency from ROIs on the renal cortex 803 and latissimus dorsi muscle 804. Error bars on the panel FIG. 8B represent physiological noise (PN). Consistent with simulation, S_{ASL} increases with the number of labeling pulses. PN are similar in all scans. S_{ASL} 805 and SNR efficiency 806 measured in renal cortex matched with simulated S_{ASL} 807 and SNR efficiency 808 results while measured S_{ASL} 809 and SNR efficiency 8010 in the latissimus dorsi muscle are within the noise level.

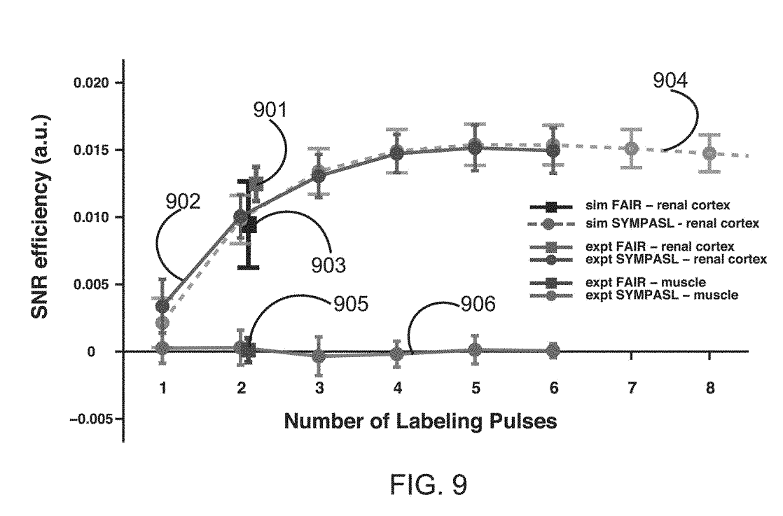

FIG. 9 shows combined SNR efficiency measured from six healthy subjects. Error bars represent standard deviation across subjects. Measured renal cortex SNR efficiency from FAIR 901 and SYMPASL 902 are in good agreement with simulation results from FAIR 903 and SYMPASL 904 while that in the latissimus doris muscle from FAIR 905 and SYMPASL 906 are within the noise level.

The computer that has been mentioned herein may include one or more processors, tangible memories (e.g., random access memories (RAMs), read-only memories (ROMs), and/or programmable read only memories (PROMS)), tangible storage devices (e.g., hard disk drives, CD/DVD drives, and/or flash memories), system buses, video processing components, network communication components, input/output ports, and/or user interface devices (e.g., keyboards, pointing devices, displays, microphones, sound reproduction systems, and/or touch screens).

The computer may include one or more components at the same or different locations. When at different locations, the components may be configured to communicate with one another through a wired and/or wireless network communication system.

The computer may include software (e.g., one or more operating systems, device drivers, application programs, and/or communication programs). When software is included, the software includes programming instructions and may include associated data and libraries. When included, the programming instructions are configured to implement one or more algorithms that implement one or more of the functions of the computer, as recited herein. The description of each function that is performed by the computer also constitutes a description of the algorithm(s) that performs that function.

The software may be stored on or in one or more non-transitory, tangible storage devices, such as one or more hard disk drives, CDs, DVDs, and/or flash memories. The software may be in source code and/or object code format. Associated data may be stored in any type of volatile and/or non-volatile memory. The software may be loaded into a non-transitory memory and executed by one or more processors.

The components, steps, features, objects, benefits, and advantages that have been discussed are merely illustrative. None of them, nor the discussions relating to them, are intended to limit the scope of protection in any way. Numerous other embodiments are also contemplated. These include embodiments that have fewer, additional, and/or different components, steps, features, objects, benefits, and/or advantages. These also include embodiments in which the components and/or steps are arranged and/or ordered differently.

One variation of SYMPASL may include labeling blood in a control and labeled mode. In the control mode, the multiple labeling pulses may have the same effects on the imaged tissues as in the labeled mode, but with minimal or without disturbance to the inflowing blood.

One variation of SYMPASL may include one or more imaging acquisitions and one or more labeling pulses every heartbeat. Labeling pulses may occur simultaneously and within imaging acquisitions.

One variation of SYMPASL may include applying multiple inversion pulses that may reduce the MR signals of tissues at the imaging locations. That may increase the SNR of SYMPASL.

One variation of SYMPASL may include continuous labeling and imaging. Acquired MR signals may be retrospectively processed to either obtain information indicative of perfusion at certain physiological condition or at various physiological conditions.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

All articles, patents, patent applications, and other publications that have been cited in this disclosure are incorporated herein by reference.

The phrase “means for” when used in a claim is intended to and should be interpreted to embrace the corresponding structures and materials that have been described and their equivalents. Similarly, the phrase “step for” when used in a claim is intended to and should be interpreted to embrace the corresponding acts that have been described and their equivalents. The absence of these phrases from a claim means that the claim is not intended to and should not be interpreted to be limited to these corresponding structures, materials, or acts, or to their equivalents.

The scope of protection is limited solely by the claims that now follow. That scope is intended and should be interpreted to be as broad as is consistent with the ordinary meaning of the language that is used in the claims when interpreted in light of this specification and the prosecution history that follows, except where specific meanings have been set forth, and to encompass all structural and functional equivalents.

Relational terms such as “first” and “second” and the like may be used solely to distinguish one entity or action from another, without necessarily requiring or implying any actual relationship or order between them. The terms “comprises,” “comprising,” and any other variation thereof when used in connection with a list of elements in the specification or claims are intended to indicate that the list is not exclusive and that other elements may be included. Similarly, an element proceeded by an “a” or an “an” does not, without further constraints, preclude the existence of additional elements of the identical type.

None of the claims are intended to embrace subject matter that fails to satisfy the requirement of Sections 101, 102, or 103 of the Patent Act, nor should they be interpreted in such a way. Any unintended coverage of such subject matter is hereby disclaimed. Except as just stated in this paragraph, nothing that has been stated or illustrated is intended or should be interpreted to cause a dedication of any component, step, feature, object, benefit, advantage, or equivalent to the public, regardless of whether it is or is not recited in the claims.

The abstract is provided to help the reader quickly ascertain the nature of the technical disclosure. It is submitted with the understanding that it will not be used to interpret or limit the scope or meaning of the claims. In addition, various features in the foregoing detailed description are grouped together in various embodiments to streamline the disclosure. This method of disclosure should not be interpreted as requiring claimed embodiments to require more features than are expressly recited in each claim. Rather, as the following claims reflect, inventive subject matter lies in less than all features of a single disclosed embodiment. Thus, the following claims are hereby incorporated into the detailed description, with each claim standing on its own as separately claimed subject matter.

The invention claimed is:

1. A magnetic resonance imaging system comprising:
a magnet;
gradient coils;
an RF pulse transmitter;
an RF receiver that receives MR signals from tissue that has been exposed to RF pulses, gradient fields, and a magnetic field; and
a computer that includes a processor that has a configuration that:
causes the RF pulse transmitter and gradient coils to emit multiple labeling pulses at predetermined labeling times directed to blood in a subject;
causes the RF pulse transmitter, gradient coils, and magnet to generate MR signals directed to tissue at one or more spatial locations within the subject that receives the blood;
causes the RF receiver to receive MR signals emitted by the tissue at predetermined imaging times;
generates an image of the tissue based on the received MR signals;
repeats the foregoing four actions one or more times; and
generates information indicative of perfusion within the tissue based on the generated images.

2. The magnetic imaging system of claim 1, further comprising a sensor that monitors a physiological activity of the subject and wherein the predetermined labeling times and predetermined imaging times are set by the computer based on the monitored physiological activity of the subject.

3. The magnetic imaging system of claim 2, wherein the physiological activity is heartbeats and the predetermined labeling times are set by the computer to occur during a user-specified cardiac phase within each heartbeat.

4. The magnetic resonance imaging system of claim 3, wherein the predetermined labeling times and the predetermined imaging times are set by the computer to occur every heartbeat.

5. The magnetic resonance imaging system of claim 1, wherein the predetermined labeling times are set by the computer to occur when the RF receiver receives MR signals emitted by the tissue.

6. The magnetic resonance imaging system of claim 2, wherein labeling pulses and imaging continuously occur.

7. The magnetic resonance imaging system of claim 2, wherein acquired MR signals are retrospectively processed based on recorded physiological activity.

8. The magnetic imaging system of claim 2, wherein the physiological activity is respiration and the predetermined labeling and imaging times are set by the computer based on a rate of the monitored respiration of the subject.

9. The magnetic imaging system of claim 2, wherein the physiological activity is blood flow and wherein the predetermined labeling times are set by the computer to occur when the monitored blood flow has a rate above a threshold.

10. The magnetic resonance imaging system of claim 2, wherein the sensor provides an ECG, PG, acoustic, optical, electromagnetic, real-time MR, CT, PET, SPECT, or ultrasound navigator signal in response to the monitored physiological activity and wherein the computer sets the labeling and imaging times in real time based on the signal provided by the sensor.

11. The magnetic resonance imaging system of claim 1 wherein the predetermined labeling and imaging times are specified by a human operator.

12. The magnetic resonance imaging system of claim 1 wherein the predetermined imaging times are set by the computer to occur immediately following completion of the multiple labeling pulses.

13. The magnetic resonance imaging system of claim 1 wherein the multiple labeling pulses are of different user-selectable types.

14. The magnetic resonance imaging system of claim 13 wherein the different user-selectable types of pulses include spatially selective, velocity selective, and acceleration selective pulses.

15. The magnetic resonance imaging system of claim 1, wherein the computer causes the RF pulse transmitter, gradient coils, and magnet to generate MR signals directed to the tissue at multiple spatial locations simultaneously.

16. The magnetic resonance imaging system of claim 1, wherein the computer causes the RF pulse transmitter, gradient coils, and magnet to generate MR signals directed to the tissue at multiple spatial locations sequentially.

17. The magnetic resonance imaging system of claim 1, wherein the computer causes the RF pulse transmitter, gradient coils, and magnet to generate MR signals directed to the tissue at multiple spatial locations in different organs.

18. The magnetic resonance imaging system of claim 1, wherein the predetermined labeling times are both before and between the predetermined imaging times.

19. The magnetic resonance imaging system of claim 1 wherein the computer generates information indicative of arterial transit time from the location of the blood to which the labeling pulses are directed to the spatial locations of tissue that receives the blood based on the images.
